(19) United States
(12) Patent Application Publication
(10) Pub. No.: US 2023/0310544 A1
(43) Pub. Date: Oct. 5, 2023

(54) ANTI-RNA VIRAL PHARMACEUTICAL COMBINATION THERAPY WITH APROTININ + anti-RNA DRUG

(71) Applicants: Alexandre Vasilievich Ivachtchenko, Hallandale, FL (US); Alena Alexandrovna Ivachtchenko, Hallandale, FL (US); Nikolay Filippovich Savchuk, Rancho Santa Fe, CA (US); ASAVI LLC, Hallandale, FL (US)

(72) Inventors: Alexandre Vasilievich Ivachtchenko, Hallandale, FL (US); Andrey Alexandrovich Ivashchenko, Moscow (RU); Alena Alexandrovna Ivachtchenko, Hallandale, FL (US); Ilya Andreevich Ivashchenko, Moscow region (RU); Nikolay Filippovich Savchuk, Rancho Santa Fe, CA (US)

(73) Assignees: ALEXANDRE VASILIEVICH IVACHTCHENKO, Hallandale, FL (US); ALENA ALEXANDROVNA IVACHTCHENKO, Hallandale, FL (US); NIKOLAY FILIPPOVICH SAVCHUK, Rancho Santa Fe, CA (US); ASAVI LLC, Hallandale, FL (US)

(21) Appl. No.: 17/709,731

(22) Filed: Mar. 31, 2022

(51) Int. Cl. A61K 38/17; A61P 31/14; A61P 31/16; A61K 31/4965; A61K 31/196; A61K 31/351

(52) U.S. Cl. CPC A61K 38/1709; A61P 31/14; A61P 31/16; A61K 31/4965; A61K 31/196; A61K 31/351

(57) ABSTRACT

A pharmaceutical combination prevention and treatment of RNA virus infection and/or a disease associated with this infection, of acute respiratory viral infections (ARVI) and severe acute respiratory syndrome (SARS), including viral and bacterial pneumonia and COVID-19, using aprotinin (APR) and an anti-RNA ingredient. A pharmaceutical kit and pharmaceutical composition in the form of an aqueous solution (APC) or a lyophilizate (PCL) for a pharmaceutical combination treatment and prevention of RNA virus infection and/or a disease associated with this infection.

### FIELD OF THE INVENTION

The present application is directed to a new pharmaceutical combination therapy with aprotinin+anti-RNA drug, a pharmaceutical composition and pharmaceutical kit intended for the prevention and treatment of RNA virus infection, including acute respiratory virus infections (ARVI) and severe acute respiratory syndrome (SARS), as well as disease caused by RNA virus infection.

### BACKGROUND OF THE INVENTION

Acute respiratory virus infections (ARVI) and severe acute respiratory syndrome (SARS) are caused by different types and groups of RNA viruses of influenza, parainfluenza, respiratory syncytial, adenoviruses and coronaviruses. The ARVI and SARS viruses have a tropism for the epithelium of the mucous membranes of the respiratory system. They are characterized by catarrhal damage to the mucous membranes of the larynx, trachea, and bronchi with involvement of the lungs in the process. The infections are transmitted mainly by aerosol transmission.

With ARVI and SARS, not complicated by a secondary bacterial infection, microscopically, changes in the respiratory system are insignificant. Inflammation of the mucous membrane of the upper respiratory tract is in the nature of catarrhal laryngotracheobronchitis, which is more pronounced with influenza. With influenza, there is hyperemia of the mucous membrane of the subglottic region, trachea and bronchi with the presence of punctate hemorrhages. In parainfluenza and MS infection, changes in the upper respiratory tract are insignificant. With all acute respiratory viral infections in the lungs, plethora, edema, foci of marginal emphysema, and often focal small subpleural hemorrhages are noted. Pneumonia with ARVI is focal in nature.

According to the National Center for Biotechnology Information (NCBI) taxonomy database, ˜70,000 influenza viruses have been identified, differing in their antigenic spectrum, including ˜53,000 influenza A virus (120 subtype), ˜16,200—influenza B virus, ˜320 influenza C virus, and ˜90 influenza D virus (Taxonomic Browser (Orthomyxoviridae)—NCBI. https://www.ncbi.nlm.nih.gov/Taxonomy/Browser/wwwtax.cgi?id=11308).

Influenza is an ARVI that affects all age groups and is associated with high mortality rates during pandemics, epidemics and sporadic outbreaks. Influenza affects about 10% of the world's population every year. The most common complications of influenza include viral or bacterial pneumonia, which mostly kill about half a million people each year.

Influenza vaccination is the most effective method for preventing influenza infection and its complications. The influenza vaccine's efficacy varies each season based on the circulating influenza strains and vaccine uptake rates based on the circulating influenza strains and vaccine uptake rates [M. Javanian et al. A brief review of influenza virus infection (J. Med. Virol. 2021, 93(8), 4638-4646. doi: 10.1002/jmv.26990. Epub 2021 Apr. 14).

The influenza virus has several targets for effective antiviral drugs. Most antiviral drugs are oral medications. The first classes of influenza drugs approved for use were adamantanes, which block the M2 ion channel on the surface of the virion. While these drugs were effective, the relatively rapid emergence of resistant strains ultimately rendered them ineffective. Oseltamivir (Tamiflu), the most common neuraminidase drug, has a similar disadvantage. Also known are polymerase and nucleoprotein inhibitors that target the replication apparatus of influenza viruses. Two of these drugs, baloxavir marboxil (Xofluza) and favipiravir (Avigan), have appeared on the market but are currently not widely used. Since 2018, the Xofluza drug has been approved for the treatment of influenza in Japan, the USA, Hong Kong, Australia, Russia, and Europe (Y. Bai et al. Antivirals Targeting the Surface Glycoproteins of Influenza Virus: Mechanisms of Action and Resistance. Viruses 2021, 13 (4), 624, 1-16. doi: 10.3390/v13040624). Examples of parenteral influenza drugs are inhaled Relenza (zanamivir) and intravenous Rapivab (peramivir).

The rapid emergence of resistant strains to known drugs and new resistant strains of influenza remains an ongoing threat and serious problem. Therefore, the development of new anti-influenza drugs remains an urgent task.

Severe Acute Respiratory Syndrome Coronavirus 2 (SARS-CoV-2) is also an RNA virus that causes COVID-19 (2019 Coronavirus Disease). SARS-CoV-2 is responsible for the ongoing COVID-19 pandemic. SARS-CoV-2 is a virus that belongs to a type of coronavirus associated with SARS-CoV (Coronaviridae Study Group of the International Committee on Taxonomy of Viruses (April 2020). “The species Severe acute respiratory syndrome-related coronavirus: classifying 2019-nCoV and naming it SARS-CoV-2”. Nat. Microbial. 2020, 5(4), 536-544. doi:10.1038/s41564-020-0695-z). This virus was first identified in December 2019 in Wuhan city, Hubei province, China. On Mar. 11, 2020, WHO declared the outbreak a public health emergency of international concern. SARS-CoV-2 is the successor to the SARS-CoV-1 virus that caused the SARS outbreak in 2002-2004 (New coronavirus stable for hours on surfaces”. National Institutes of Health (NIH). NIH.gov. 17 Mar. 2020. Archived from the original on 23 Mar. 2020. Retrieved 4 May 2020). SARS-CoV-2 has undergone many changes in two years, and each new mutation has been more perfect than the previous one. First discovered in India in December 2020, the Delta mutation is spreading across continents at an alarming rate. Delta penetrates lung cells more easily than the original virus (the virus that circulated in the early stages of the pandemic). In addition, the Delta strain is more effective in combining infected lung cells with uninfected ones. This could contribute to the more severe course of COVID-19. It is currently the predominant variant of SARS-CoV-2 worldwide. Delta is believed to be more than twice as infectious as previous SARS-CoV-2 variants (K. Katella. 5 Things to Know About The Delta Variant. Yale Medicine Nov. 19, 2021. https://www.yalemedicine.org/nMws/5-things-to-know-delta-variant-covid). The new variant of coronavirus Omicron was detected in laboratories in Botswana and South Africa on 22 Nov. 2021. The variant has an unusually large number of mutations, several of which are novel and a significant number of which affect the spike protein targeted by most COVID-19 vaccines at the time of discovering the Omicron variant. This level of variation has led to concerns regarding its transmissibility, immune system evasion, and vaccine resistance. Omicron spreads faster than any previously known variant. As of Dec. 17, 2021, 77 countries have now reported cases of Omicron, and “the reality is that Omicron is probably in most countries, even if it hasn't been detected yet (L. Smith-Spark, What can the world learn from countries where Omicron is surging? CNN Fri Dec. 17, 2021. https://www.cnn.com/2021/12/17/health/covid-omicron-what-can-the-world-learn-cmd-intl/index.html).

As of Feb. 18, 2022, 418 650 474 confirmed cases of people infected with coronavirus were registered in the world, of which 5 856 224 unfortunately have died (Coronavirus disease (COVID-19). (WHO 2021. https://www.who.int/emergencies/diseases/novel-coronavirus-2019?adgroupsurvey={adgroupsurvey}&gclid=EAIaIQobChMIzbzIp5ii9AIVFD6tBh0VXAGIEAA YAiAAEgJOvvD_BwE).

Vaccination remains one of the main public health interventions to combat ARVI and SARS-CoV-2. However, vaccine development times of at least six months limit their applicability during outbreaks of new strains of RNA viruses, in particular influenza and SARS-CoV-2 viruses. Therefore, anti-RNA viral drugs represent important measures against new RNA strains, including influenza viruses and coronaviruses.

Previous attempts to provide an anticoronavirus drug include a pharmaceutical combination therapy of SARS-CoV-2, and disease associated with this infection (COVID-19), using aprotinin (APR), hydroxychloroquine and/or ivermectin and/or bafilomycin. (U.S. Ser. No. 11/007,187 (2021).

However, the effectiveness of this medicine (pharmaceutical composition) has not been proven, and moreover, at present, hydroxychloroquine, ivermectin and bafilomycin included in the claims of U.S. Ser. No. 11/007,187 are not recommended for the prevention and treatment of SARS-CoV-2/COVID-19.

Hydroxychloroquine is a drug used to treat malaria, rheumatoid arthritis, and lupus erythematosus. The mechanism of action of hydroxychloroquine is to increase the pH inside intracellular vacuoles and change processes such as the cleavage of proteins by acid hydrolases in lysosomes, assembly of macromolecules in endosomes, and post-translational modification of proteins in the Golgi apparatus (R. I. Fox. Mechanism of action of hydroxychloroquine as an antirheumatic drug. Semin. Arthritis Rheum. 1993, 23(2), Suppl. 1, 82-91. https://doi.org/10.1016/S0049-0172(10)80012-5). In June 2020, the FDA revoked authorization for the emergency use of hydroxychloroquine and chloroquine phosphate for the treatment of COVID-19. WHO and NIH have also discontinued studies evaluating hydroxychloroquine for the treatment of COVID-19 due to lack of benefit (Medically reviewed by Drugs.com. An Update: Is hydroxychloroquine effective for COVID-19? Last updated on Sep. 4, 2021. https://www.drugs.com/medical-answers/hydroxychloroquine-effective-covid-19-3536024/). Hydroxychloroquine phosphate and sulfate do not improve the clinical status of patients hospitalized with COVID-19 (A. B. Cavalcanti et al. Hydroxychloroquine with or without Azithromycin in Mild-to-Moderate Covid-19. N. Engl. J. Med. 2020, 383, 2041-2052. W. H. Self et al. Effect of Hydroxychloroquine on Clinical Status at 14 Days in Hospitalized Patients with COVID-19. JAMA 2020, 324, 2165-2176).

Ivermectin is used to treat infections with certain parasitic worms and head lice, as well as skin conditions such as rosacea. The mechanism of action is ivermectin an agonist of the neurotransmitter gamma-aminobutyric acid (GABA). It disrupts GABA-mediated neurosynaptic transmission in the central nervous system. Ivermectin selectively and with high affinity binds to glutamate-driven chloride ion channels in invertebrate muscles and microfilament nerve cells. This binding causes an increase in the permeability of the cell membrane for chloride ions and leads to hyperpolarization of the cell, which leads to paralysis and death of the parasite (Ivermectin: Uses, Interactions, Mechanism of Action. DrugBank 2005, Updated at Nov. 17, 2021. https://go.drugbank.com/drugs/DB00602). FDA has neither approved nor approved the use of ivermectin to prevent or treat COVID-19 in humans or animals. (FDA. Why You Should Not Use Ivermectin to Treat or Prevent COVID-19. FDA 09/03/2021. https://www.fda.gov/consumers/consumer-updates/why-you-should-not-use-ivermectin-treat-or-prevent-covid-19). Ivermectin maker Merck claims there is “no significant evidence of clinical activity or efficacy in patients with Covid-19.” FDA has warned people not to use this drug to treat Covid-19. (P. G. Lurie. Ivermectin for Covid-19: abundance of hype, dearth of evidence. STAT Aug. 25, 2021. https://www.statnews.com/2021/08/25/ivermectin-for-covid-19-abundance-of-hype-dearth-of-evidence/).

Bafilomycin is an antibiotic. Its mechanism of action is to disrupt autophagic flow, independent inhibition of V-ATPase-dependent acidification and Ca-P60A/SERCA-dependent autophagosome-lysosome fusion (C. Mauvezin et al. Autophagosome-lysosome fusion is independent of V-ATPase-mediated acidification. Nat. Commun. 2015, 6, 7007. http://dx.doi.org/10.1038/ncomms8007). Antibiotics are not recommended for the prevention and treatment of coronavirus infection, as antibiotics only treat bacterial infections (https://www.who.int/ru/emergencies/diseases/novel-coronavirus-2019/advice-for-public/myth-busters?gclid=EAIaIQobChMItda7hfnR8wIV_w2tBh0hjg1eEAAYASAAEg K2aPD_BwE#antibiotics).

The main disadvantage of this pharmaceutical combination therapy is the absence of experimental data confirming the effectiveness of this therapy, and the use as active ingredients (hydroxychloroquine and/or ivermectin and/or bafilomycin), which have serious side effects and do not improve the clinical status of patients infected with SARS-Cov-2/COVID-19.

Previous attempts to provide an anticoronavirus drug also include a method of treating of moderate COVID-19 patients by the intravenous aprotinin (APR) and oral Avifavir® combination therapy. This therapy is more effective because primary and secondary efficacy endpoints of therapy by the APR+FVP combination significantly better than efficacy endpoints of therapy by the individual components (Table 1). (Ivashchenko A. A. et al. Effect of Aprotinin and Avifavir® Combination Therapy for Moderate COVID-19 Patients. Viruses 2021, 13, 1253. https://doi.org/10.3390/v13071253.).

TABLE 1
Primary and secondary efficacy endpoints of therapy by the intravenous
aprotinin (APR), oral Avifavir ® and their combination.

| Primary and Secondary | | | | |
|---|---|---|---|---|
| Efficacy Endpoints | APR + SOC | FVP + SOC) | | APR + FVP + SOC |
| Median time to elimination of SARS- | 7.5 (6-9) | 4.5 | (4-9) | 3.5 (3-4) |
| CoV-2 confirmed by RT-PCR, days (IQR) | | | | |
| Median time to normalization of CRP | 6.0 (6-6) | 14.0 | (5.5-14) | 3.5 (3-5) |
| concentration (≤10 mg/L) in patient's | | | | |
| blood, days (IQR) | | | | |
| Median time to normalization of D-dimer | 4.5 (3-6) | NA | | 5.0 (4-5) |
| concentration (<253 ng/ml) in patient's | | | | |
| blood, days (IQR) | | | | |
| Median time to normalization of body | 3.0 (2-3) | 2.0 | (1-3) | 1.0 (1-3) |
| temperature (<37° C.), days (IQR) | | | | |
| Median time to improvement in clinical | 11.0 (6-11) | 14.0 | (11.5-16) | 5.0 (5-5) |
| status by 2 points on the WHO-OSCI, | | | | |
| days (IQR) | | | | |

The main disadvantage of this pharmaceutical combination therapy is the use of two drugs (oral FVP and intravenous APR) with different routes their administration to the patients. This greatly complicates the treatment of patients with moderate to severe of COVID-2.

The aim of the present application is the elimination of the above disadvantages inherent in the known pharmaceutical combination therapies of SARS-CoV-2 and COVID-19.

### Terms Used in the Description

The term “drug” (also called medicine, medicament, pharmaceutical drug, or medicinal drug) refers to a drug used to diagnose, cure, treat, or prevent disease and means a substance (or a mixture of substances in the form of a pharmaceutical composition).

The term “oral drug” refers to solutions, powders, tablets, capsules, and pills that are taken by mouth and swallowed.

The term “parenteral drug” refers to drugs that are into the body bypassing the gastrointestinal tract. Parenteral drugs are solutions for injection, inhalation, and sprays, including for nasal or drip application, and other finished dosage forms, in this case intended for the treatment and prevention of viral infections and diseases caused by them.

The term “pharmaceutical composition” as used herein means a composition comprising at least two active ingredients (substances), namely aprotinin an inhibitor of RNA viruses, and at least one excipient. This pharmaceutical composition is intended primarily for parenteral administration of drugs into the body, in which they bypass the gastrointestinal tract, in contrast to the oral route of drug administration. These are primarily intravenous, inhalation and nasal routes of drug administration.

The term “parenteral pharmaceutical composition (PPC)” is intended for parenteral administration of drugs into the body of a patient. These are primarily intravenous, inhalation and nasal routes of drug administration.

The term “excipient” as used herein refers to a compound that is used to prepare a pharmaceutical composition and is generally safe, non-toxic, and neither biologically nor otherwise undesirable, and includes excipients that are acceptable to humans and animals. The present application uses primarily excipients selected from the series: water, sodium chloride, L-lysine monohydrate, 2-hydroxy-beta-cyclodextrin, betadex sulfobutyl ether sodium, sodium hydroxide, hydrochloric acid, benzyl alcohol, ethanol, glycerin, dimethyl sulfoxide, peppermint oil, 1,1,1,2-tetrafluoroethane, and some others.

The term “pharmaceutical kit” as used herein means a kit including at least two drags: anti-RNK drag or saline solution of its active ingredient, or lyophilizate of its active ingredient and the parenteral drug Trasylol®, or Gordox®, or Aprotex®, or Antagosan®, or Contrycal®, or Traskolan®, or others parenteral drugs including APR, or aqueous or saline solution containing APR.

The term “pharmaceutical combination therapy (treatment)” is therapy that uses more than one pharmaceutical medication (drug). “Pharmaceutical combination therapy (treatment)” may be achieved by prescribing/administering separate drugs, or dosage forms that contain more than one active ingredient (such as fixed-dose combinations).

The term “combination therapy (treatment)” is therapy that uses more than one medication or modality. Typically, the term refers to using multiple therapies to treat a single disease, and often all the therapies are pharmaceutical. “Pharmaceutical” combination therapy may be achieved by prescribing/administering separate drugs, or, where available, dosage forms that contain more than one active ingredient (such as fixed-dose combinations).

The term “therapeutically effective amount” or “dose” as used herein means the amount of medicine needed to reduce the symptoms of a disease in a patient. The dose of medicine will be tailored to the individual requirements in each case. This dose can vary widely depending on numerous factors, such as the severity of the patient's illness, the age and general health of the patient, other drugs with which the patient is being treated, the method and form of administration of medicine, and the experience of the attending physician. Typically, treatment is started with a large initial “loading dose” to rapidly reduce or eliminate the virus and followed by tapering the dose to a level sufficient to prevent an outbreak of infection.

The term “patient” means a mammal including but not limited to humans, cattle, pigs, sheep, chickens, turkeys, buffaloes, llamas, ostriches, dogs, cats, hamsters, and mice, preferably the patient is a human.

The term “active ingredient (substance)” as used herein means aprotinin and an inhibitor of RNA viruses used in a pharmaceutical composition or drug.

The term “parenteral therapies” are administration of drugs is primarily injections (intravenously, into the muscles, under the skin), inhalations and nasally (spray, drops).

### SUMMARY OF THE INVENTION

The first aspect of the present application relates to a pharmaceutical combination treatment and prevention of RNA virus infection and/or a disease associated with this infection, including viral and bacterial pneumonia and COVID-19, using aprotinin (APR) and an anti RNA ingredient or a drug containing one of these ingredients.

Another aspect of the present application is a pharmaceutical kit for a pharmaceutical combination treatment and prevention of RNA virus infection and/or a disease associated with this infection, including viral and bacterial pneumonia and COVID-19, consisting of the parenteral drug Trasylol®, or Gordox®, or Aprotex®, or Antagosan®, or Contrycal®, or Traskolan®, or other parenteral drug that include APR, or an aqueous or saline solution containing APR, and an anti RNA drug selected from a number:
the drug Avigan, or Areplivir, or Coronavir, or Remdeform, or their analogue, including AVF as an active ingredient, excluding Avifavir, or a solution for infusion, which includes FVP (T-705) and excipients, or a lyophilizate for infusion, which includes FVP and excipients, if necessary;
the drug containing as an active ingredient AV5080 or a pharmaceutically acceptable salt thereof or a solution for infusion, which includes AV5080, or lyophilizate or powder for infusion, which includes AV5080 or a pharmaceutically acceptable salt thereof and excipients, if necessary;
the drug Tamiflu or their analogue or an aqueous or saline solution for infusion, which includes oseltamivir (OS) or a pharmaceutically acceptable salt thereof and excipients, if necessary, or lyophilizate or powder for infusion, which includes oseltamivir (OS) or a pharmaceutically acceptable salt thereof and excipients, if necessary;
the drug Rapivab or their analogue or an aqueous or saline solution for infusion, which includes peramivir (PRV) or a pharmaceutically acceptable salt thereof and excipients, if necessary, or lyophilizate or powder for infusion, which includes peramivir (PRV) or a pharmaceutically acceptable salt thereof and excipients, if necessary;
the drug Relenza or their analogue or an aqueous or saline solution for infusion, which includes zanamivir (ZA) or a pharmaceutically acceptable salt thereof and excipients, if necessary, or lyophilizate or powder for infusion, which includes zanamivir (ZA) or a pharmaceutically acceptable salt thereof and excipients, if necessary.

Another aspect of the present application is a pharmaceutical composition in the form of an aqueous solution (APC) or a lyophilizate (PCL) which includes APR, an anti RNA active ingredient selected from a number: AV5080 or a pharmaceutically acceptable salt thereof or oseltamivir (OS) or a pharmaceutically acceptable salt thereof or peramivir (PRV) or a pharmaceutically acceptable salt thereof or zanamivir (ZA) and excipients for a pharmaceutical combination treatment and prevention of RNA virus infection and/or a disease associated with this infection, including viral and bacterial pneumonia and COVID-19.

The excipients are selected from the series: water, sodium chloride, L-lysine monohydrate, 2-hydroxy-beta-cyclodextrin, sodium hydroxide, hydrochloric acid, benzyl alcohol, ethanol, glycerin, dimethyl sulfoxide, peppermint oil, 1,1,1,2-tetrafluoroethane, and others.

An anti RNA drug is selected from the group including favipiravir (FVP), AV5080, oseltamivir (OS), peramivir (PRV), and zanamivir (ZA).

APR is a natural protease inhibitor with a long history of clinical use since the 1960s and a good safety profile. APR, under the trade names Trasylol, Gordox, and others, is used as an intravenous medication given by injection to reduce bleeding during complex surgeries such as heart and liver surgery (https://www.rxlist.com/trasylol-drug.htm #indications; https://yandex.ru/health/pills/product/gordoks-203), as an antiviral drug for the treatment and prevention of respiratory pathology of viral or viral-bacterial origin (U.S. Pat. No. 5,723,439). A number of pharmaceutical compositions containing APR are known to be used for the treatment of various diseases.

The parenteral drugs Trasylol®, Gordox®, Aprotex®, Antagosan®, Contrycal®, Traskolan®, other a parenteral drug that include APR, or an aqueous or saline solution containing APR with an APR activity of 5000-10000 KIU/ml containing excipients selected from the series: sodium chloride, sodium hydroxide, 1M hydrochloric acid solution, benzyl alcohol, and others. These drugs are used to prevent intraoperative blood loss and reduce the volume of blood transfusion in liver and heart transplants, coronary artery bypass grafting using a heart-lung machine in adult patients who are at increased risk of bleeding or need blood transfusion. These drugs are also recommended as a preventive treatment for patients who are likely to be at an increased risk of bleeding or need transfusion (https://www.vidal.by/poisk_preparatov/gordox.html).

Known drugs containing APR as an active ingredient are parenteral drugs administered to patients intravenously or by inhalation.

APR is effective in preventing infection by viruses influenza and parainfluenza by inhibiting protease activity. Influenza-associated viruses also use this protease activation process for entry into the host cell (Zhirnov O. P. et al. Aprotinin and similar protease inhibitors as drugs against influenza. Antiviral Res. 2011, 92(1), 27-36. Shen L. W. et al. TMPRSS2: A potential target for treatment of influenza virus and coronavirus infections. Biochimie. 2017, 142, 1-10. doi: 10.1016/j.biochi.2017.07.016.).

APR inhibits the entry of SARS-CoV-2 into cells (M. Hoffmann et al. SARS-CoV-2 Cell Entry Depends on ACE2 and TMPRSS2 and Is Blocked by a Clinically Proven Protease Inhibitor. Cell 2020, 181 (2), 271-280. e8. doi: 10.1016/j.cell.2020.02.052), inhibits the replication of SARS-CoV-2 (D. Bojkova et al. Aprotinin Inhibitors SARS-CoV-2 Replication. Cells 2020, 9 (11), 2377. https: //doi.org/10.3390/cells9112377), and can be used for the prevention and treatment of SARS-CoV-2/COVID-19 (RU 2738885) and respiratory pathology of viral or viral-bacterial origin (U.S. Pat. No. 5,723,439) including influenza (RU 2054180).

FVP is an anti-viral agent that selectively and potently inhibits the RNA-dependent RNA polymerase (RdRp) of RNA viruses (Y. Furuta, T. Komeno, T. Nakamura. Favipiravir (T-705), a broad spectrum inhibitor of viral RNA polymerase. Proc. Jpn. Acad. Ser. B Phys. Biol. Sci. 2017, 93(7), 449-463). FVP proved to be the first effective oral anti-SARS-CoV-2 drug. FVP is active against a wide range of RNA viruses including influenza virus, rhinovirus, and respiratory syncytial virus. FVP is more effective than OS (Tamiflu) in the treatment of influenza infections, OS-resistant viruses, and pathogenic avian influenza A(H5N1) and A(H7N9) viruses. These influenza strains cause more severe illness, especially pneumonia, than seasonal influenza, and the mortality rate from influenza A(H5N1) is 53.5% and from influenza A(H7N9), 34% (K. Shiraki, T. Daikoku. FVP, an anti-influenza drug against life-threatening RNA virus infections. Pharmacol. Ther. 2020, 209.107512. doi: 10.1016/j.pharmthera. 2020.107512). In vitro, the 50% effective concentration (EC₅₀) of FVP against SARS-CoV-2 is 61.88 μM/L in Vero E6 cells. FVP has high potential for treating COVID-19 patients. In vitro, the 50% effective concentration (EC₅₀) of FVP against SARS-CoV-2 is 61.88 μM/L in Vero E6 cells (P. Eloy et al. Dose rationale for Favipiravir use in patients infected with SARS-CoV-2. Clin. Pharmacol Ther. 2020, 108(2), 188. https://doi.org/10.1002/cpt.1877). FVP induces viral clearance within 7 days and contributes to clinical improvement within 14 days. The results showed that favipiravir has great potential for treating COVID-19, especially in patients with mild to moderate illness. (T. Manabe et al. Favipiravir for the treatment of patients with COVID-19: a systematic review and meta-analysis. BMC Infectious Diseases 2021, 21, 489. https://doi.org/10.1186/s12879-021-06164-x).

In February 2020, FVP was approved for clinical trials as a drug for the treatment of COVID-19. (Gamal El-Din A. Abuo-Rahma et al. Potential repurposed SARS-CoV-2 (COVID-19) infection drugs. RSC Adv., 2020, 10, 26895. doi:10.1039/d0ra05821a).

FVP is produced and supplied in powder form (https://www.indiamart.com/proddetail/favipiravir-api-22403040530.html) or as lyophilisate to prepare a concentrate for an infusion solution consisting of 400 mg/vial or 800 mg/vial FVP and 192 mg/vial g or 384 mg/vial of L-lysine monohydrate, respectively, and sodium hydroxide with a pH under 6.8-8.0 (https://medum.ru/areplivir-ukoly-inekcii).

AV5080 is a neuraminidase inhibitor, oral drug candidate, an inhibitor of influenza A and B neuraminidase. According to preclinical studies, its activity was similar or higher than that of zanamivir (Relenza®) or oseltamivir (Tamiflu®). AV5080 effectively inhibits the neuraminidase activity of oseltamivir-resistant influenza A virus strains (New candidate for oral anti-influenza drug AV5080. Antimicrobial Chemotherapy Advance Access published Apr. 11, 2014). AV5080 has been shown to be effective and safe in phase 2 clinical trials (https://clinicaltrials.gov/ct2/show/NCT05095545?term=NCT05095545&draw=2&rank=1) and is currently in phase 3 (https://clinicaltrials.gov/ct2/show/NCT05093998?term=AV5080&draw=2&rank=1).

OS phosphate (Tamiflu®) is an antiviral medication used to treat and prevent influenza A and influenza B. OS was approved for medical use in the US in 1999. It was the first influenza neuraminidase inhibitor available by mouth. A 2014 Cochrane Review concluded that OS does not reduce hospitalizations and that there is no evidence of reduction in complications of influenza. Two meta-analyses have concluded that benefits in those who are otherwise healthy do not outweigh its risks. They also found little evidence regarding whether treatment changes the risk of hospitalization or death in high-risk populations. However, another meta-analysis found that oseltamivir was effective for prevention of influenza at the individual and household levels (Oseltamivir. Wikipedia This page was last edited on 11 Oct. 2021. https://en.wikipedia.org/wiki/Oseltamivir).

PRV (Rapivab®) is an intravenous anti-RNA viral drug developed by BioCryst Pharmaceuticals for the treatment of influenza. PRV is a neuraminidase inhibitor acting as a transition-state analogue inhibitor of influenza neuraminidase and thereby preventing new viruses from emerging from infected cells (https://www.accessdata.fda.gov/drugsatfda_docs/label/2014/2064261b1.pdf; https://www.tga.gov.au/sites/default/files/auspar-peramivir-181105.pdf).

ZA (Relenza®) is an oral inhalation drug for the prevention and treatment of influenza in patients aged 7 years and older who have had symptoms of influenza A and B for more than 2 days. This influenza neuraminidase inhibitor was developed by the Australian biotech firm Biota Holdings.

It was licensed by Glaxo in 1990 and approved in the US in 1999 for use only as a cure for the flu. In 2006, it was approved for the prevention of influenza A and B (https://www.mdpedia.net/view_htmlphp?sq=Obama%20 Care & lang=en & q=Zanamivir; https://www.accessdata.fda.gov/drugsatfda_docs/label/2010/021036s025lbl.pdf).

The inventors of the present application unexpectedly found that the active ingredients (APR and an inhibitor of viral RNA) included in the new anti-RNA viral APC do not interact with each other during the preparation, short-term storage, and use of the composition as a parenteral (intravenous, inhalation, or intranasal) drug.

Another aspect of the present application is directed to anti-RNA viral parenteral drug, which is the APC of the present application.

The parenteral drug (an aqueous pharmaceutical composition (APC)) according to the present application is intended for the prevention and treatment of RNA virus infection and/or a disease associated with this infection, including viral and bacterial pneumonia and COVID-19, especially for the prevention and treatment of influenza viruses and coronaviruses, mainly for the prevention and treatment of influenza and SARS-CoV-2 and/or a disease associated with this infections, including viral and bacterial pneumonia and COVID-19.

Another aspect of the present application is directed to the containing the aqueous APC of the present application container.

Another aspect of the present application is directed to an ampoule and a bottle containing the aqueous APC of the present application.

Another aspect of the present application is directed to an inhaler (nebulizer) selected from a number of compressor or ultrasonic or electronic mesh inhaler (nebulizer) containing the aqueous APC of the present application.

Another aspect of the present application is directed to a pocket or portable inhaler (nebulizer) containing the aqueous APC of the present application.

Another aspect of the present application is directed to a container, which is a can for nasal spray containing the aqueous APC of the present application.

Another aspect of the present application is directed to the prevention and/or treatment of RNA viral infection and/or a disease associated with this infection, including viral and bacterial pneumonia and COVID-19, especially for the prevention and treatment of influenza viruses and coronaviruses, mainly for the prevention and treatment of influenza and SARS-CoV-2 and/or a disease associated with this infections, including viral and bacterial pneumonia and COVID-19, in a patient by administration to the patient of the drugs from a pharmaceutical kit in a therapeutically effective amount once or twice a day.

In accordance with the present application, patients are treated by intravenous administration of with the infusion drugs from pharmaceutical kit of the present application in a therapeutically effective amount once or twice a day (as prescribed by a physician, depending on the patient's condition).

In accordance with the present application, patients are treated by intravenous administration of drug including APR and oral drug from pharmaceutical kit of the present application in a therapeutically effective amount once or twice a day (as prescribed by a physician, depending on the patient's condition).

In accordance with the present application, prophylaxis of patients is carried out with a therapeutically effective amount of a nasal spray, which is APC of the present application.

In accordance with the present application, the prophylaxis is carried out using balloons for nasal spray for the nose and throat into the nose (into the nasopharynx) and throat 3-6 times a day into each nostril and into the throat—about 1 ml per session and about 3-6 ml per day).

In accordance with the present application is used the spray balloons for the nose and throat [https://sm-sprayer.en.made-in-china.com/product-group/tbonLPBJsUVW/Nasal-sprayer-1.html?gclid=EAIaIQobChMImduuj7K-9AIVPjytBh3jkQ0xEAAYBCAAEgKY1fD_BwE].

In accordance with the present application, the prophylaxis and treatment of patients is carried out by inhalation using ultrasonic nebulizers 3-6 times a day for 5-7 days a therapeutically effective amount of APC of the present application.

According to the present application is used Portable ultrasonic inhalers and nebulizers [https://www.feellife.com/aerogo-liquid-cup; https://www.google.com/search?q=portable+ultrasonic+nebulizer&oq=Portable+ultrasonic+inhalers+nebulizers&aqs=edge.1.69i57j0i33313j69i64.5076j0j4&sourceid=chrome&ie=UTF-8].

Another aspect of the present application is directed to the use of APC or the parenteral drug of the present application for the prevention and/or treatment of the RNA viral infection and/or disease associated with this infection by intravenous, or inhalation, or intranasal administration to a patient of APC or the parenteral drug of the present application in a therapeutically effective amount.

Another aspect of the present application is directed to the use of APC or the parenteral drug of the present application in a therapeutically effective amount for the prevention and/or treatment of the acute respiratory viral infection, or a coronavirus infection.

Another aspect of the present application is directed to the use of APC or the parenteral drug of the present application in a therapeutically effective amount for the prevention and/or treatment of influenza and/or pneumonia associated with influenza or SARS-CoV-2 and/or COVID-19 associated with SARS-CoV-2.

The use of a new APC or the parenteral drug containing two active ingredients greatly simplifies the process of prophylaxis and treatment of patients in comparison with combination therapy.

Another aspect of the present application is directed to a method of obtaining APC or the parenteral drug of the present application by dissolving APR, an anti RNA ingredient, and excipients in a 0.9% aqueous sodium chloride solution.

Another aspect of the present application is directed to a method of obtaining the aqueous APC or the parenteral drug of the present application by dissolving a lyophilizate containing APR, an anti RNA ingredient, and excipients in saline (0.9% aqueous sodium chloride solution).

The active ingredients and the anti RNA viral composition of the present application are best kept their activity in lyophilized form. Before use, a lyophilizate is dissolved in saline to obtain the dosage forms for intravenous, inhalation, or intranasal administration.

Another aspect of the present application is directed to the use of APR in the form of a powder, or a lyophilisate, or a concentrate, or a drug selected from the group consisting of Trasylol®, Gordox®, Aprotex®, Antagosan®, Contrycal®, Traskolan®, other a parenteral drug that include APR for preparing APC or the parenteral drug of the present application.

Another aspect of the present application is directed to the use of an anti RNA ingredient in the form of a powder, or a lyophilisate, or a concentrate for preparing the APC or the parenteral drug of the present application.

Another aspect of the present application is directed to the use of an anti RNA ingredient in the form of a powder, or a lyophilisate, or a concentrate and APR as Trasylol®, or Gordox®, or Aprotex®, or Traskolan®.

APC or the parenteral drug for intravenous, inhalation, or intranasal administration can be obtained, including immediately before use, by sequential dissolution in physiological solution of crystalline APR or its lyophilisate, for example, Contrykal, a crystalline an anti RNA ingredient or its lyophilizate, for example, Areplivir (FVP lyophilisate), and, if necessary, excipients.

APC or the parenteral drug for intravenous, inhalation, or intranasal administration can be obtained, including immediately before use, by dissolving a crystalline an anti RNA ingredient or its lyophilizate in an aqueous solution of APR or in known drugs that are aqueous solutions of APR, for example, Trasylol®, Gordox®, Aprotex®, Antagosan®, Contrycal®, Traskolan®, other a parenteral drug that include APR, and, if necessary, bringing the resulting compositions to the required concentration of active ingredients with a 0.9% aqueous sodium chloride solution.

The new APC or the parenteral drug for intravenous, inhalation, and intranasal administration can be obtained, including immediately before use, by sequential dissolution in physiological solution of crystalline APR or its lyophilizate (e.g. Contryka), a crystalline an anti RNA ingredient or its lyophilizate (e.g. Areplivir—favipiravir lyophilizate) and excipients, if necessary

The new APC or the parenteral drug for intravenous, inhalation, and intranasal administration can be obtained, including immediately before use, by dissolving a crystalline inhibitor of viral RNA or its lyophilisate in an aqueous solution of APR or in known drugs that are aqueous solutions of APR, e.g. Trasylol®, Gordox®, Aprotex®, Antagosan®, Contrycal®, Traskolan®, other a parenteral drug that include APR, and, if necessary, by bringing the resulting compositions to the required concentration of active ingredients with a 0.9% aqueous sodium chloride solution.

The use of the new APC or the parenteral drug containing APR and an anti RNA ingredient according to the present application greatly simplifies the prevention and improves efficacy treatment of patients infected with ARVI and/or coronavirus and the moderate and severe patients with diseases caused by these RNA viruses.

According to the present application, replacing the combination treatment of patients with oral+parenteral drugs with one parenteral drug (APC) of the present application halves the number of preventive and/or therapeutic procedures and allows effective prevention of coronavirus and/or ARVI and treatment especially patients with moderate and severe diseases caused by coronavirus and/or ARVI.

According to the present application, replacing the combination treatment of patients with oral+parenteral drugs with one parenteral drug (APC) of the present application halves the number of preventive and/or therapeutic procedures and allows effective prevention of coronavirus and/or ARVI and treatment especially patients with moderate and severe with diseases caused by coronavirus and/or ARVI.

After intranasal administration of APC according to the present application to SARS-CoV-2 infected Syrian hamsters significantly reduces the SARS-CoV-2 RNA titer. In particular, in comparison with the control group, the intranasal administration of the drug of the present application to infected hamsters already 3 days after administration radically reduces the titer of SARS-CoV-2 RNA in nasal washings and clears the tissues of lung of animals from SARS-CoV-2 RNA. This means that the prophylactic drug treatment of the present application prevented the multiplication and spread of SARS-CoV-2 to the lungs.

Prevention and treatment of mice by inhaling effectively protected the animals from death increasing their average lifespan and reliably suppressing virus multiplication in the lungs of animals (by an average of 31gTCID₅₀/0.1 ml compared with control group).

After intraperitoneal treatment for 4 days with APC 1.12 to the transgenic mice (B6.Cg-Tg(K18-ACE2)2Prlmn/HEMI Hemizygous for Tg(K18-CE2)2Prlmn from Jackson Immunoresearch, West Grove, PA, USA) infected with mouse-adapted SARS-CoV-2 (“Dubrovka” strain, identification number GenBank: MW161041.1) a statistically significant reduction in virus titer by more than an order of magnitude was obtained in the lungs of infected animals, compared to the control group of infected but untreated animals.

After intraperitoneal treatment for 4 days with APC 1.10 to the Syrian hamsters weighing 100-120 g (State Scientific Center for Virology and Biotechnology “Vector” of Rospotrebnadzor, Russia) infected with SARS-CoV-2 (strain hCoV-19/Australia/VIC01/2020) a statistically significant reduction in virus titer by order of magnitude was obtained in the lungs of infected animals, compared with the control group of infected but untreated animals.

Below are examples of the preparation and use of APC, confirming but not limiting the present application.

### EXAMPLE 1. PREPARATION OF THE APC'S COMPRISING APR AND FVP (APC 1.1-1.12)

APC 1.1. FVP (175 g) from Zenji Pharmaceuticals (Suzhou) Ltd, China, and APR (92.6 mg, 500,000 KIU) with an activity of 5400 KIU/mg from Wanhua Biochem, China, were dissolved with stirring in saline (50 ml) to yield APC 1.1 containing 3.5 mg/ml FVP and 10,000 KIU/ml APR.

PPC 1.2. 1 ml APC 1.1 was added with stirring to 159 ml of saline to yield APC 1.2 containing 21.9 μg/ml FVP and 62.5 KIU/ml APR.

APC 1.3. 1 ml PPC 1.1 was added with stirring to 399 ml of saline to yield APC 1.3 containing 8.75 μg/ml FVP and 25 KIU/ml APR.

APC 1.4. 925.9 mg APR (powder) from Wanhua Biochem, China, with an activity of 5400 KIU/mg (total 5,000,000 KIU of APR), 3000 mg lysine and 6000 mg FVP (powder) were dissolved with stirring in 500 ml of saline. A 10M aqueous NaOH solution was added to pH 7.08±7.6 to yield 500 ml of APC 1.4 containing 10,000 KIU/ml APR and 12 mg/ml FVP for intravenous, nasal (spray), and inhalation treatment and prevention of RNA viral infections.

APC 1.5. 40 ml saline was slowly added along the inner wall of a bottle containing 600 mg FVP lyophilisate and 300 mg L-lysine monohydrate. The bottle was shaken vigorously until the drug was completely dissolved. The resulting FVP solution was added with stirring to a mixture of 50 ml Trasylol®, or Gordox®, or Aprotex®, or Traskolan® containing 500,000 KIU of APR and 110 ml saline to yield 200 ml of APC 1.5 containing FVP (3.0 mg/ml) and APR (2500 KIU/ml).

APC 1.6. A mixture of a lyophilisate containing 350 mg FVP, 46.3 mg (250,000 KIU) APR, and 180 mg L-lysine monohydrate was dissolved with vigorous stirring in 250 ml of saline to yield 250 ml of APC 1.6 containing FVP (1.4 mg/ml) and APR (1000 KIU/ml).

APC 1.7. A mixture of 148.1 mg APR powder from Wanhua Biochem, China, with an activity of 5400 KIU/mg (total 800000 KIU of APR), 300 mg L-lysine, and 600 mg FVP (powder) was dissolved with stirring in 100 ml of saline. A 10M aqueous NaOH solution was added to pH 7.08±7.6 to yield ×100 ml of APC 1.7 containing APR (8000 KIU/ml) and FVP (6 mg/ml).

APC 1.8. 650 mg lyophilisate of FVP, 88 mg (475,000 KIE) APR, and 360 mg L-lysine monohydrate was dissolved with vigorous stirring in 250 ml of saline to yield 500 ml of APC 1.8 containing FVP (1.3 mg/ml) and APR (950 KIU/ml).

APC 1.9. 50 mg lyophilisate of FVP, 92.6 mg (500,000 KIU) APR and of 36 mg L-lysine monohydrate was dissolved with vigorous stirring in 50 ml of saline to yield 50 ml of APC1.9 containing FVP (1.0 mg/ml) and APR (10000 KIU/ml).

APC 1.10. 400 mg FVP and 192 mg L-lysine monohydrate was added with vigorous stirring to a mixture of 20 ml Gordox containing 10000 KIU/ml of APR. A 10M aqueous NaOH solution (260 μl) was added to pH 7.08÷7.6 with vigorous stirring until complete dissolution of FVP to yield 20 ml of APC 1.10 containing FVP (20.0 mg/ml) and APR (10000 KIU/ml).

APC 1.11. 20 mg FVP and 10 mg L-lysine monohydrate was added with vigorous stirring to a mixture of 20 ml Gordox containing 10000 KIU/ml of APR. A 10M aqueous NaOH solution was added to pH 7.08±7.6 with vigorous stirring until complete dissolution of FVP to yield 20 ml of APC1.11 containing FVP (1.0 mg/ml) and APR (10000 KIU/ml).

APC 1.12. 1000 mg FVP and 500 mg L-lysine monohydrate was added with vigorous stirring to a mixture of 20 ml Gordox containing 10000 KIU/ml of APR. A 10M aqueous NaOH solution was added to pH 7.08±7.6 with vigorous stirring until complete dissolution of FVP to yield 20 ml of APC 1.12 containing FVP (50 mg/ml) and APR (10000 KIU/ml).

### EXAMPLE 2. THE STABILITY OF PPC 1.3 FROM EXAMPLE 1

The stability of APC 1.3 from Example 1 was studied by UV spectroscopy on an Agilent 8453 spectrophotometer after storage under normal conditions and under stress tests. The optical densities of UV absorption band maxima of APC 1.3 after stress test conditions strongly differ from the original spectrum of APC 1.3. (Table 2).

The percentage of change in optical density (A) under conditions 2-5 compared to optical density under Conditions 1 is 9-90% (Table 2). This indicates that APC 3 and other PPC's from Example 1 are limitedly stable under rapid tests conditions and them must be used within a few hours after preparation.

TABLE 2
Optical density at the maxima of absorption bands in the UV
spectra of APC 1.3 immediately after preparation of their
solutions and after their exposure for 48 hours: in the light
at 25° C. (2), in the dark at 30° C. and 65% humidity
(3), in the dark at 40° C. and 75% humidity (4), and
in the dark at 60° C. and 60% humidity (5). Δ, % is
the percentage of change in optical density under Conditions
2-5 compared to optical density under Conditions 1.

| Maxima in UV spectra and percent change in | |
|---|---|
| absorbance (Δ) under Conditions 2-5 compared | |
| to absorbance under Conditions 1 | |
| 230 nm | 263 nm |

| | Optical | | Optical | |
|---|---|---|---|---|
| Conditions | density | Δ, % | density | Δ, % |
| 1 | 0.42199 | — | 0.29439 | — |
| 2 | 0.33316 | −21.05 | 0.19785 | −32.79 |
| 3 | 0.38285 | −9.28 | 0.25172 | −14.49 |
| 4 | 0.31597 | −25.12 | 0.18999 | −35.46 |
| 5 | 0.11929 | −71.73 | 0.02899 | −90.15 |

### EXAMPLE 3. PREPARATION OF THE APC COMPRISING APROTININ AND AV5080 (APC 3.1-3.4)

APC 3.1. 50 mg AV5080 and 90.19 mg (487,000 KIU)APR from Wanhua Biochem, China with an activity of 5400 KIU/mg were dissolved in a mixture of 35 ml a 20% solution of 2-hydroxy-beta-cyclodextrin and 15 ml saline under ultrasonic stirring for 25 minutes to yield 50 ml of APC 3.1 containing 1.0 mg/ml AV5080 and 9740 KIU/ml APR.

APC 3.2. 50 mg AV5080 and 90.19 mg (487,000 KIU) APR from Wanhua Biochem, China with an activity of 5400 KIU/mg were dissolved in a mixture of 350 ml 20% solution of 2-hydroxy-beta-cyclodextrin and a 150 ml saline under ultrasonic stirring for 25 minutes to yield 500 ml of APC 3.2 containing AV5080 (0.1 mg/ml) and APR (9974 KIU/ml).

APC 3.3. 99 ml Saline was added under ultrasonic stirring to 1 ml APC 3.1 to yield 100 ml of PPC 3.3 containing AV5080 (10 μg/ml) and APR (97.4 KIU/ml).

APC 3.4. 2.5 mg AV5080 and 92.6 mg (500,000 KIU) APR from Wanhua Biochem China with an activity of 5400 KIU/mg were dissolved in 50 ml saline under ultrasonic stirring for 15 minutes to yield 50 ml of APC 3.4 containing AV5080 (0.05 mg/ml) and APR (10,000 KIU/ml).

### EXAMPLE 4. PREPARATION OF APC 4.1 CONTAINING APR AND PRV

APC 4.1. 7.2 mg PRV from Ambeed, USA and 2.22 mg (12,000 KIU) APR from Wanhua Biochem, China with an activity of 5400 KIU/mg were dissolve ed in 200 ml saline to yield 200 ml of APC 4.1 containing PRV (0.36 mg/ml) and APR (11.0511 g/ml, 60 KIU/ml).

The assessment of optical density changes in the UV spectra of APC 4.1 (Table 4) when stored for 48 hours under selected conditions indicates moderate stability of APC 4.1. The percentage of change in optical density (A) under conditions 4 and 5 compared to optical density under Conditions 1 is >2%. This indicates that PPC 4.1 is limitedly stable under rapid tests conditions and APC 4.1 must be used within a few hours after preparation.

TABLE 4
Optical density at the maxima of absorption bands in the UV spectra
of APC 4.1 component immediately after solution preparation
composition 1 and after exposure for 48 hours in the light at
25° C. (2), in the dark at 25° C. (3), in the dark
at 3-5° C. (4), and in the dark at 60° C. (5). Δ, %
is the percentage of change in optical density under Conditions
2-5 compared to optical density under Conditions 1.

Maxima in UV spectra and percent change in
absorbance (Δ) under Conditions 2-5 compared
to absorbance under Conditions 1
203 nm

| | Optical | |
|---|---|---|
| Conditions | density | Δ, % |
| 1 | 1.0728 | — |
| 2 | 1.0813 | 0.79 |
| 3 | 1.0879 | 1.41 |
| 4 | 1.0509 | −2.04 |
| 5 | 1.0979 | 2.34 |

### EXAMPLE 5. PREPARATION OF APC 5.1 COMPRISING APR AND ZA

APC 5.1. 7.0 mg of ZA from Ambeed (USA) and 7.41 mg (40000 KIU) of APR from Wanhua Biochem (China) with an activity of 5400 KIU/mg were dissolved in 50 ml of saline to yield 50 ml APC 5.1 containing 0.14 mg/ml ZA and 800 KIU/ml APR.

The results of evaluating the change in optical density in the UV spectra of APC 5.1 (Table 5), when stored for 48 hours under the selected conditions, indicate moderate stability of APC 5.1. The results obtained show that anti-RNA viral PPC 5.1 must be used within a few hours after preparation.

TABLE 5
Optical density at the maxima of absorption bands in the
UV spectra of APC 5.1 immediately after preparation of
their solutions (composition 1) and after exposure of
their for 48 hours in the light at 25° C. (2), in
the dark at 25° C. (3), in the dark at 3-5°
C. (4), and in the dark at 60° C. (5). Δ, % is
the percentage of change in optical density under Conditions
2-5 compared to optical density under Conditions 1.

| Maxima in UV spectra and percent change in | |
|---|---|
| absorbance (Δ) under Conditions 2-5 compared | |
| to absorbance under Conditions 1 | |
| 245 nm | 277 nm |

| | Optical | | Optical | |
|---|---|---|---|---|
| Conditions | density | Δ, % | density | Δ, % |
| 1 | 0.30778 | — | 0.12895 | — |
| 2 | 0.30476 | −0.98 | 0.12538 | −2.77 |
| 3 | 0.30836 | 0.19 | 0.12912 | 0.13 |
| 4 | 0.30698 | −0.26 | 0.12755 | −1.09 |
| 5 | 0.3062 | −0.51 | 0.12635 | −2.02 |

### EXAMPLE 6. A DEVICE FOR INHALATION THERAPY AND PREVENTION OF RNA VIRAL INFECTIONS

To prepare a device for inhalation therapy and prevention of RNA viral infections, 5-10 ml APC 1.2 from Example 1 containing FVP (21.9 μg/ml) and APR (62.5 KIU/ml) is placed into a compression nebulizer Omron NE-C300 Complete or in a portable ultrasonic mesh nebulizer, for example, Feellife Aerogo mesh nebulizer.

### EXAMPLE 7. DEVICES FOR NASAL SPRAY THERAPY AND PREVENTION OF RNA VIRAL INFECTIONS

To prepare a device for nasal spray therapy and prevention of RNA viral infections, 5-10 ml APC 1.6 from Example 1 containing FVP (1.4 mg/ml) and APR (1000 KIU/ml) or APC 3.2 from Example 3 containing AV5080 (0.1 mg/ml) and APR (974 KIU/ml) is placed into a plastic can for nasal.

### EXAMPLE 8. PREVENTION AND TREATMENT OF SYRIAN HAMSTERS INFECTED WITH THE SARS-COV-2 USING AN ANTI-RNA VIRAL PHARMACEUTICAL COMPOSITION CONTAINING APROTININ (APR) AND AN INHIBITOR OF THE RNA VIRUS

The efficacy of the viral pharmaceutical composition of the present application was evaluated using a model of SARS-CoV-2 infection in Syrian hamsters [R. Boudewijns et al. STAT2 signaling as double-edged sword restricting viral dissemination but driving severe pneumonia in SARS-CoV-2 infected hamsters. BioRxiv preprint. doi: https://doi. org/10.1101/2020.04.23.056838; this version posted Apr. 24, 2020. https://www.biorxiv.org/content/10.1101/2020.04.23.056838v1].

The strain SARS-CoV-2 nCoV/Victoria/1/2020 was obtained from the State Research CenRNeasyter of Virology and Biotechnology VECTOR (Russia). The infectious virus was isolated by sequential passage in Vero E6 cells. The titer of the viral suspension was determined by endpoint dilution on Vero E6 cells using the Reed-Muench method. The work related to the live virus was carried out under isolated laboratory conditions that meet the international BSL-3+VECTOR requirements.

Vero E6 cells from VECTOR's Collection of Cell Cultures were cultured in Minimum Essential Medium (MEM) (Gibco) supplemented with 10% fetal bovine serum (Integro), 1% L-glutamine (Gibco), and 1% Bicarbonate (Gibco). Endpoint titrations were performed with a medium containing 2% fetal bovine serum.

APR, Gordox, and FVP included in the anti-RNA viral pharmaceutical composition were obtained from commercial sources: APR from Wanhua Biochem (China), Gordox from Sotex PharmFirm (Russia), FVP from Zenji Pharmaceuticals (Suzhou) Ltd. (China).

Wild-type Syrian hamsters at the age of 6-10 months from CrolInfo Ltd. Russia were kept with unlimited access to food and water. Hamsters were randomized into two cohorts, 8 animals in each cohort (4 males and 4 females).

Hamsters were anesthetized with zoletil-xyla and inoculated into each nostril with 50 μl anesthetic combination containing 10³TCID₅₀.

Animals of control cohort 1 were injected with water for injection. Animals of cohort 2 were injected into each nostril with 50 μl of APC 1.7 from Example 1 containing APR (8000 KIU/ml) and FVP (6 mg/ml) twice a day for 3 days at a daily dose of 16000 KIU for APR and 1.2 mg for FVP.

First injection 1 hour before infection. On Day 4, the hamsters were euthanized by intravenous administration of 500 μl (200 mg/ml) sodium pentobarbital (Vetoquinol SA). The clinical condition of the lungs was evaluated, lung tissues were harvested, and viral RNA was quantitatively determined using real-time PCR. During the study, the body weights and temperature of the animals were assessed on a daily basis.

Nasopharyngeal lavages were taken every day using MEM medium (1 ml). Hamster lung tissues were harvested after sacrifice and homogenized using a Precellys homogenizer in a 350 RNeasy lysis buffer (RNeasy Mini kit, Qiagen) and centrifuged (10,000 rpm, 5 min) to remove cell debris. RNA was extracted according to the manufacturer's instructions. Real-time PCR was performed on the LightCycler96 platform (Roche) using the iTaq Universal Probes One-Step RT-qPCR kit (BioRad) [R. Boudewijns et al. STAT2 signaling as double-edged sword restricting viral dissemination but driving severe pneumonia in SARS-CoV-2 infected hamsters. BioRxiv preprint. doi: https://doi.org/10.1101/2020.04.23.056838; this version posted Apr. 24, 2020. https://www.biorxiv.org/content/10.1101/2020.04.23.056838v1].

For histological analysis, lung tissue was fixed in 4% formaldehyde, embedded in paraffin, and stained with hematoxylin-eosin. Damage was assessed on a scale from 1 to 3: stagnation, intra-alveolar bleeding, apoptotic bodies in the bronchial epithelium, necrotic bronchiolitis, perivascular edema, bronchopneumonia, perivascular inflammation, peribronchial inflammation, and vascular inflammation.

Statistical analysis was performed using the GraphPed Prism software from GraphPed Software, Inc. Statistical significance was determined using the Mann-Whitney nonparametric U-test. The values of P≤0.05 were considered significant.

An analysis of the results obtained showed that APC 1.7 comprising APR and a RNA virus inhibitor demonstrated a high anti-SARS-CoV-2/COVID-19 efficacy. Thus, in comparison with the control group, 3 days after intranasal administration of the drug, the titer of SARS CoV-2 RNA in both nasopharyngeal washes and in the tissues of the lungs decreased about 105 times. This means that prophylactic drug treatment prevented SARS-CoV-2 from multiplying and spreading in the lungs.

### EXAMPLE 9. PREVENTION AND TREATMENT OF MICE INFECTED WITH INFLUENZA A/CALIFORNIA/2009 (H1N1) PDM09 VIRUS BY INHALATION WITH AN ANTI-RNA VIRAL PHARMACEUTICAL COMPOSITION IN A MODEL OF INFLUENZA PNEUMONIA

In the experiment, 3 groups of BALB/c female mice (from Andreevka nursery, Russia) weighing 12-14 g were formed, 13 animals per group, of which 10 mice were tested for survival and 3 mice were tested for the titer of the virus in the lungs.

Group 1—prophylaxis of mice according to Scheme 1:
on day 0 the mice received 3 (morning, afternoon, and evening) inhalations of APC 1.6 from Example 1;
in the morning of day 1, the mice received drug inhalation, then after 1 hour they were infected with the influenza A/California/2009(H1N1) pdm09 virus, and in the afternoon and evening they received inhalations again;
on days 2-10, the mice received 3 (morning, afternoon, and evening) drug inhalations a day.

Group 2—treatment of mice according to Scheme 2:
in the morning of day 1, the mice received drug inhalation, then after 1 hour they were infected with the influenza A/California/2009(H1N1) pdm09 virus, and in the afternoon and evening they received two more inhalations;
on days 2-10, the mice received 3 (morning, afternoon, and evening) drug inhalations a day.

Group 3—untreated mice: in the morning of day 1, the mice were infected with the influenza virus, and then water for injection was intragastrically administered immediately after infection and in the evening of the same day.

Mice randomized into groups were infected intranasally with the influenza A/California/2009 (H1N1) pdm09 virus sourced from WHO and adapted for mice under light anesthesia at a dose of 5 MLD₅₀/ml (25 μl in each nostril, 10^{4.5} TCID₅₀/0.1 ml).

For treatment, APC 1.6 containing FVP (1.4 mg/ml) and APR (1000 KIU/ml).

APC 1.6 was administered by inhalation using a nebulizer. For this purpose, a group of 5 animals was placed in a chamber and given inhalation of 2 ml of the test drug for 10 minutes.

The treated and control animals were monitored daily for 16 full days (from the moment the animals were infected with the influenza virus). Mortality was recorded daily in both groups.

Euthanasia (painless killing of the animal) was carried out by the responsible person in accordance with the existing ethical requirements by dislocation of the cervical vertebrae with preliminary anesthesia with ether. Euthanasia was performed promptly after the end of the experiments.

The activity of the compounds in the mouse model of influenza pneumonia was assessed according to the following criteria: animal survival, increase in average life expectancy, dynamics of weight loss, and a decrease in the lung virus titers after 24 and 96 hours.

The mortality rate was determined as the ratio of dead to infected in the group.

The average life span of animals was calculated from the total number of observation days (after infection) according to the formula: MSD=Σf*(d−1)/n, where f is the number of mice that died on day d; surviving mice, for which day d is the last day of observation, are also taken into account; and n is the number of mice in the group. For example, there were 10 mice in a group, of which 1 mouse died on the 8th day, 3 mice died on the 10th day, 1 mouse died on the 12th day, and 5 mice survived. The experiment lasted 14 days. In this case, the average life expectancy of animals calculated according to the above formula will be: MSD=Σf*(d−1)/n=[1*(8−1)+3*(10−1)+1*(12−1)+5*(14−1)]/10=11 days.

A statistically significant increase in the survival rate of animals (p<0.05), an increase in their lifespan, a statistically significant decrease in the viral titer in the lungs of infected animals (on average, >1.75 log TCID₅₀) after the introduction of drugs were compared with a control group of infected untreated animals.

The digital data obtained was statistically processed using the Statistica 8.0 software. Comparison of survival in the groups of mice was performed by means of one-way analysis of variance (ANOVA) using the Statistica 8.0 software.

The results obtained are presented in Table 1, from which it can be seen that the prevention of group 1 and the treatment of group 2 with APC 1.6 from Example 1 provide high efficacy in infected mice's
survival rate (70-80%),
life expectancy (12.6-13.4 days),
life elongation compared to control group 1 (46.5-55.8%), and
the maximum decrease in the virus titer in the lungs for 4 days after infection (lgTCID₅₀/0.1 ml=(2.08±0.14)−(3.1±2.84) compared to control group 3.

Inhalation prophylaxis and treatment of mice by anti-RNA viral APC1.6 from Example 1 effectively protected animals from death increasing their average lifespan and reliably inhibiting virus multiplication in the lungs, as compared to the viral control group, by an average of 3 lgTCID₅₀/0.1 ml (Table 6).

Thus it was shown the inhalation prophylaxis and treatment of mice by APC 1.6 from Example 1 effectively protected animals from death increasing their average lifespan and reliably inhibiting virus multiplication in the lungs, as compared to the control group, by an average of 3 lgTCID₅₀/0.1 ml (Table 6).

TABLE 6
Efficacy of the prevention and treatment of influenza pneumonia in mice infected
with the influenza A/California/2009(H1N1) pdm09 virus adapted to mice with
APC 1.6 from Example 1 containing APR and the RNA polymerase inhibitor FVP
(group 1 - prevention, group 2 - treatment, group 3 - placebo).

| | Survival | | Life | | | |
|---|---|---|---|---|---|---|
| | Alive/ | | expectancy | Life | lgTCID₅₀/0.1 ml^{d} | |
| Group | Dead | Mortality, % | (days) | elongation, %^{c} | 1 day | 4 days |
| 1 | 8/2^{a} | 20 | 13.4 | 55.8 | 2.9 ± 2.66 | 2.7 ± 2.49 |
| 2 | 7/3^{b} | 30 | 12.6 | 46.5 | 3 ± 2.74 | 3.1 ± 2.84 |
| 3 | 1/10 | 90 | 8.6 | — | 6.2 ± 0.91 | 5.6 ± 0.82 |

^{a}p = 0.000538);
^{b}p = 0.004103;
^{c}compared to control group 3;
^{c}virus titer 1 and 4 days after infection.

### EXAMPLE 10. TREATMENT OF MICE INFECTED WITH INFLUENZA A/CALIFORNIA/04/2009 (H1N1) VIRUS ADAPTED TO MICE BY INTRAPERITONEAL INJECTION OF APC IN AN INFLUENZA PNEUMONIA MODEL

In the experiment, 6 groups of BALB/c female mice (Stezar nursery, Russia) weighing 12-14 g were formed, 13 animals per group, of which 10 mice were tested for survival and 3 mice were tested for the titer of the virus in the lungs.
Group 1—control group, untreated mice: in the morning of day 1, the mice were infected with the influenza virus, and then water for injection was intragastrically administered immediately after infection and in the evening of the same day.
Group 2—treatment with a saline APR solution (10 000 KIU/ml). APR dose: 50 000 KIU/kg, (600±700) KIU/mouse, (0.06±0.07) ml/mouse of saline APR solution (10 000 KIU/ml).
Group 3—treatment with a saline AV5080 0.05 mg/ml solution.
   AV5080 dose: 0.25 mg/kg, (0.003±0.0035) mg/mouse, (0.06±0.07) ml/mouse of saline solution.
Group 4—treatment with APC 3.4 from Example 3 containing AV5080 (0.05 mg/ml) and APR (10 000 KIU/ml). AV5080+APR dose: 0.25 mg/kg+50 000 KIU/kg, (0.003±0.0035) mg/mouse+(600±700) KIU/mouse, (0.06±0.07) ml/mouse of pharmaceutical composition 1-9.
Group 5—treatment with a saline FVP solution (1.0 mg/ml). FVP dose: 5.0 mg/kg, (0.06±0.07) mg/mouse, (0.06±0.07) ml/mouse of saline FVP solution.
Group 6—treatment with APC 1.11 from Example 1 containing FVP (1.0 mg/ml) and APR (10 000 KIU/ml). FVP+APR dose: 5.0 mg/kg+50 000 KIU/kg, (0.06±0.07) mg/mouse+(600±700) KIU/mouse, (0.06±0.07) ml/mouse of PPC 1.11.
Groups 2-6 were treated according to Scheme 1:
   in the morning of day 1, the drug was administered to mice intraperitoneally immediately after infection with the influenza A/California/2009 (H1N1) virus and in the evening (˜8-12 hours after infection);
   days 2-5: treatment 2 times a day. Monitoring the survival of mice: 15 days. Measuring the titer of the virus in the lungs (3 mice per group): on day 6 after the last administration of the drug;
   monitoring the survival of mice: 16 days.

Mice randomized into groups were infected intranasally with influenza A/California/04/2009 (H1N1) virus sourced from WHO and adapted for mice under light anesthesia at a dose of 5MLD₅₀/ml (25 μl in each nostril—10^{4.5} TCID₅₀/0.1 ml).

Euthanasia (painless killing of the animal) was carried out by the responsible person in accordance with the existing ethical requirements by dislocation of the cervical vertebrae with preliminary ether anesthesia. Euthanasia was performed promptly after the end of the experiments.

The activity of the compounds in the mouse model of influenza pneumonia was assessed according to the following criteria: animal survival, increase in average life expectancy, dynamics of weight loss, decrease in the titer of the virus in the lungs after 5 days.

The mortality rate was determined as the ratio of dead to infected in the group.

The average life span of animals was calculated from the total number of observation days (after infection) according to the formula: MSD=Σf*(d−1)/n, where f is the number of mice that died on day d; surviving mice, for which day d is the last day of observation, are also taken into account; and n is the number of mice in the group. For example, there were 10 mice in a group, of which 1 mouse died on the 8th day, 3 mice died on the 10th day, 1 mouse died on the 12th day, and 5 mice survived. The experiment lasted 14 days. In this case, the average life expectancy of animals calculated according to the above formula will be: MSD=Σf*(d−1)/n=[1*(8−1)+3*(10−1)+1*(12−1)+5*(14−1)]/10=11 days.

After administration of the drugs, a statistically significant increase in the survival rate of animals (p<0.05), an increase in their life expectancy and a statistically significant decrease in the titer of the virus in the lungs of infected animals (on average >1.75 log TCID₅₀) were observed compared with the control group of infected untreated animals.

The digital data obtained was statistically processed using the Statistica 8.0 software. Comparison of survival in the groups of mice was performed by means of one-way analysis of variance (ANOVA) using the Statistica 8.0 software. The results are shown in Tables 7 and 8.

As can be seen from Table 7, the treatment of group 4 with APC 3.4 provides high efficacy in infected mice's
survival rate (80%),
life expectancy (14 days),
life elongation compared to control group 1 (94.2%),
the maximum decrease in the virus titer in the lungs (lgTCID₅₀/0.1 ml=2.5±0.87) compared to control group 1 and groups 2 and 3 treated with PPC3.4 containing AV5080 and APR.

An even higher efficacy is observed in the treatment of group 6 with APC 1.11 containing APR and FVP compared to control (untreated mice, group 1) and with groups 2 and 5 (APR and FVP).

As can be seen from Table 8, the treatment of group 6 with APC 1.11 also provides high efficacy in infected mice's:
survival rate (100%),
life expectancy (16 days),
life elongation compared to the control group 1 (110.5%),
the maximum decrease in the virus titer in the lungs (lgTCID₅₀/0.1 ml=2.08±0.14) compared to control group 1 and groups 2 and 5 treated with APC 1.11 containing FVP and APR.

The results obtained showed that the treatment of mice infected with influenza A/California/04/2009 (H1N1) virus adapted to mice in an influenza pneumonia model by intraperitoneal injection of APC 3.4 (Table 7) and APC 1.11 (Table 8) provides higher efficacy than the active ingredients contained in these compositions.

TABLE 7
Effectiveness of APC 3.4, its active ingredients (AV5080, APR), and the control
(untreated mice) in an influenza pneumonia model upon infection of mice
with the influenza A/California/04/2009 (H1N1) virus adapted to mice.

| | | Survival | | Life | | |
|---|---|---|---|---|---|---|
| # | | Alive/ | | expectancy | Life | |
| group | Drug^{a} | Dead | Mortality, % | (days) | elongation, %^{b} | lgTCID₅₀/0.1 ml^{c} |
| 1 | Untreated | 0/10 | 100 | 7.6 | — | >7 |
| | mice | | | | | |
| 2 | APR | 3/7 | 70 | 10.7 | 40.8 | 4.75 ± 0.43 |
| 3 | AV5080 | 7/3 | 30 | 13.0 | 71.1 | 3.92 ± 0.8 |
| 4 | APC 3.4 | 8/2 | 20 | 14.0 | 94.2 | 2.5 ± 0.87 |

^{a}Doses of drugs: 50,000 KIU/kg of APR in groups 2 and 4; 0.25 mg/kg of AV5080 in groups 3 and 4;
^{b}compared to the control group 1;
^{c}Virus titer 5 days after infection.

TABLE 8
Effectiveness of APC 1.11, its active ingredients (APR and FVP), and the control
(untreated mice) in an influenza pneumonia model upon infection of mice with
the influenza A/California/04/2009 (H1N1) virus adapted to mice.

| | | Survival | | Life | | |
|---|---|---|---|---|---|---|
| # | | Alive/ | | expectancy | Life | |
| group | Drug^{a} | Dead | Mortality, % | (days) | elongation, %^{b} | lgTCID₅₀/0.1 ml^{c} |
| 1 | Untreated | 0/10 | 100 | 7.6 | — | >7 |
| | mice | | | | | |
| 2 | APR | 3/7 | 70 | 10.7 | 40.8 | 4.75 ± 0.43 |
| 5 | FVP | 6/4 | 40 | 12.4 | 63.2 | 2.67 ± 0.29 |
| 6 | APC 1.11 | 9/0 | 0 | 16.0 | 110.5 | 2.08 ± 0.14 |

^{a}Doses of drugs: 50,000 KIU/kg of APR in groups 2 and 6; 5 mg/kg of FVP in groups 5 and 6; group 6 - untreated mice;
^{b}compared to the control group 1;
^{c}Virus titer 5 days after infection.

### EXAMPLE 11. TREATMENT WITH APC OF TRANSGENIC MICE INFECTED WITH MOUSE-ADAPTED SARS-COV-2

In the experiment, 4 groups of transgenic mice (B6.Cg-Tg(K18-ACE2)2Prlmn/HEMI Hemizygous for Tg(K18-CE2)2Prlmn from Jackson Immunoresearch, West Grove, PA, USA), females, age—6-8 weeks, weighing 19-24 g, were formed, 4 animals per group.
Group 1—control group, untreated mice: on day 1 in the morning the mice were infected with SARS-CoV-2, and then 5 ml/kg of water for injection was intragastrically administered immediately after infection and in the evening of the same day.
Group 2—treatment with Gordox—10 000 KIU/ml of APR. Dose: 50 000 KIU/kg APR. FVP;
Groups 3—treatment with saline solution of FVP—50 mg/ml. Dose: 250 mg/kg of
Groups 4—treatment with APR dose: 50 000 KIU/kg (Gordox 10000 KIU/ml of APR)++FVP dose: 250 mg/kg (Dissolve 50 mg FVP+24 mg L-lysine monohydrate in 1 ml saline, sonicate, add ˜25 μl 10N NaOH, sonicate until dissolved and pH 7-8).

Treatment regimen for transgenic mice: parenteral (intraperitoneal) administration of drugs 2 times a day; day 0-1 hour before infected with mouse-adapted SARS-CoV-2 (“Dubrovka” strain, identification number GenBank: MW161041.1) and 6-8 hours after infection; days 1, 2, 3-2 times a day, for a total of 4 days (days 0, 1, 2, 3); Day 4—lung sampling from all animals to assess the virus titer in the lungs, visual assessment of the lungs and transfer of the lungs for histology; days 0-4—daily assessment of body weight and condition of mice.

On day 0, animals from all groups were infected with the SARS-CoV-2 “Dubrovka” virus (1035 TCID₅₀/ml). All mice were infected intranasally under light ether anesthesia in a volume of 30 μl for both nostrils.

Euthanasia (painless killing of the animal) was carried out by the responsible person in accordance with the existing ethical requirements, by dislocation of the cervical vertebrae with preliminary anesthesia with ether. Euthanasia was performed promptly after the end of the experiments.

On day 4 post-infection with the virus, the animals in each group were sacrificed and the lungs were removed under sterile conditions. One lung was fixed in formalin for further histology, the second lung was prepared to measure the virus titer. To do this, after washing three times in a solution of 0.01 M phosphate buffered saline (PBS), the lungs were homogenized and resuspended in 1 ml of cold sterile PBS. The suspension was cleared from cell debris by centrifugation at 2000 g for 10 min, and the supernatant was used to determine the infectious titer of the virus in cell culture and to perform PCR. The obtained samples were stored at −80° C. until the experiments were carried out.

To determine the infectious titer of the virus from the lungs of mice, Vero CCL81 cells were seeded in 96-well Costar plates with an average density of 20,000 cells per well and grown in DMEM medium in the presence of 5% fetal calf serum, 10 mM glutamine and antibiotics (penicillin 100 IU/well). ml and streptomycin 100 μg/ml) until a complete monolayer is formed (within 3 days). Before infection with the virus, the cell culture was washed twice with DMEM medium without serum. 10-fold dilutions of each lung virus sample were prepared from 10-1 to 10-7. The prepared dilutions in a volume of 200 μL were added to cell culture plates and incubated in 5% CO2 at 37° C. for 5 days until a cytopathic effect (CPE) appeared in virus control cells. Accounting for the result of the manifestation of CPP in cells was carried out using a quantitative MTT test. The virus titer was calculated using the Ramakrishnan M. A formula in the Excel program [M. A. Ramakrishnan. Determination of 50% endpoint titer using a simple formula. World J. Virol. 2016, 5, 85-86. doi: 10.5501/wjv.v5.i2.85] and expressed as lgTCID₅₀/ml (TCID₅₀—The median tissue culture infectious dose is defined as the dilution of a virus required to infect 50% of a given cell culture.) [I. Leneva et al. Antiviral Activity of Umifenovir In Vitro against a Broad Spectrum of Coronaviruses, Including the Novel SARS-CoV-2 Virus. Viruses 2021, 13(8): 1665. doi: 10.3390/v13081665]. Next, the average titer value for samples from mice of the same group was calculated. The obtained digital data were subjected to statistical processing in the “Statistica 8.0” software.

The effectiveness of the drugs in a model of the transgenic mice infected with mouse-adapted SARS-CoV-2 was assessed according to decrease in the titer of the virus in the lungs of animals after 4 days. Parenteral (intraperitoneal) treatment of mice resulted in a reduction in virus titer (TCID₅₀/ml) in the lungs of infected animals compared to a control group of infected but untreated animals more than 13 times was observed.

### EXAMPLE 12. INTRAVENOUS TREATMENT OF SYRIAN HAMSTERS INFECTED WITH THE SARS-COV-2 USING THE APC 1.10 FROM EXAMPLE 1 CONTAINING MOV AND APR

The efficacy of the APC 1.10 of the present application was evaluated using a model of SARS-CoV-2 infection in Syrian hamsters [R. Boudewijns et al. STAT2 signaling as double-edged sword restricting viral dissemination but driving severe pneumonia in SARS-CoV-2 infected hamsters. BioRxiv preprint. doi: https://doi.org/10.1101/2020.04.23.056838; this version posted Apr. 24, 2020. https://www.biorxiv.org/content/10.1101/2020.04.23.056838v1].

The strain SARS-CoV-2 hCoV-19/Australia/VIC01/2020 was obtained from the State Research Center of Virology and Biotechnology VECTOR (Russia). The infectious virus was isolated by sequential passage in Vero E6 cells. The titer of the viral suspension was determined by endpoint dilution on Vero E6 cells using the Reed-Muench method. The work related to the live virus was carried out under isolated laboratory conditions that meet the international BSL-3+VECTOR requirements.

Vero E6 cells from VECTOR's Collection of Cell Cultures were cultured in Minimum Essential Medium (MEM) (Gibco) supplemented with 10% fetal bovine serum (Integro), 1% L-glutamine (Gibco), and 1% Bicarbonate (Gibco). Endpoint titrations were performed with a medium containing 2% fetal bovine serum.

Wild-type Syrian hamsters at the age of 6-10 months weighing 100-120 g from State Scientific Center for Virology and Biotechnology “Vector” of Rospotrebnadzor (Russia) were kept with unlimited access to food and water. Hamsters were randomized into 4 cohorts, 8 animals in each cohort (4 males and 4 females).

Hamsters were anesthetized with zoletil-xyla and inoculated into each nostril with 50 μl anesthetic combination containing 10³TCID₅₀.
Group 1—control group, untreated hamsters. Dose: 5 ml/kg saline.
Group 2—treatment with Gordox—10 000 KIU/ml of APR. Dose: 10000 KIU/kg APR.
Groups 3—treatment with saline solution of FVP—20 mg/ml. Dose: 100.0 mg/kg of MOV.
Groups 4—treatment with APC 1.10 (FVP—20 mg/ml+10000 KIU/ml of APR) from Example 1. Dose: 50 000 KIU/kg of APR+100 mg/kg of FVP.

The drugs were injected under light isoflurane anesthesia intravenously, 2 times a day for 4 days, starting the first injection one hour before infection, 6 hours after infection, then for 3 days after 12 hours.

Hamsters were checked daily for appearance, behavior and weight. On the 4th day after infection, the hamsters were euthanized by intravenous injection of 500 μl doletal (200 mg/ml sodium pentobarbital, Vétoquinol SA). Hamster lung tissues were harvested after sacrifice and homogenized using a Precellys homogenizer in a 350 μl RNeasy lysis buffer (RNeasy Mini kit, Qiagen) and centrifuged (10,000 rpm, 5 min) to remove cell debris. RNA was extracted according to the manufacturer's instructions. Real-time PCR was performed on the LightCycler96 platform (Roche) using the iTaq Universal Probes One-Step RT-qPCR kit (BioRad) [R. Boudewijns et al. STAT2 signaling as double-edged sword restricting viral dissemination but driving severe pneumonia in SARS-CoV-2 infected hamsters. BioRxiv preprint. doi: https://doi.org/10.1101/2020.04.23.056838; this version posted Apr. 24, 2020. https://www.biorxiv.org/content/10.1101/2020.04.23.056838v1].

For histological analysis, lung tissue was fixed in 4% formaldehyde, embedded in paraffin, and stained with hematoxylin-eosin. Damage was assessed on a scale from 1 to 3: stagnation, intra-alveolar bleeding, apoptotic bodies in the bronchial epithelium, necrotic bronchiolitis, perivascular edema, bronchopneumonia, perivascular inflammation, peribronchial inflammation, and vascular inflammation.

Statistical analysis was performed using the GraphPed Prism software from GraphPed Software, Inc. Statistical significance was determined using the Mann-Whitney nonparametric U-test. The values of P≤0.05 were considered significant.

An analysis of the results obtained showed that APC 1.10 comprising FVP and APR demonstrated a high anti-SARS-CoV-2/COVID-19 efficacy. Thus, in comparison with the control group, after intravenous treatment of Syrian hamsters infected with the SARS-CoV-2 of APC 1.10, the titer of SARS CoV-2 in the tissues of the lungs decreased by more than an order of magnitude compared to the control group.

1. A method for treatment of a severe acute respiratory syndrome coronavirus 2 (SARS-CoV-2) infection and/or coronavirus disease 2019 (COVID-19) comprising:
Administering a therapeutically effective amount of aprotinin and favipiravir in combination once or twice per day via inhalation or nasal spray.

2. (canceled)

3. The method of claim 1,
wherein the aprotinin and the favipiravir are administered to a mammal in a therapeutically effective amount.

4-24. (canceled)
